SEQUENCE LISTING (8 sequences)

SEQ ID NO: 1 - DNA, 18 bp, Artificial Sequence
  synthetic
gtcagccgatgcattcct

SEQ ID NO: 2 - DNA, 18 bp, Artificial Sequence
  synthetic
tcctgggagcacgtcttg

SEQ ID NO: 3 - DNA, 18 bp, Artificial Sequence
  synthetic
tgcagcatccacaacacc

SEQ ID NO: 4 - DNA, 21 bp, Artificial Sequence
  synthetic
aaggtcttccacttcccaatg

SEQ ID NO: 5 - DNA, 18 bp, Artificial Sequence
  synthetic
ctacggtgcagagttgct

SEQ ID NO: 6 - DNA, 21 bp, Artificial Sequence
  synthetic
tcttgatgatctgaggctgga

SEQ ID NO: 7 - DNA, 18 bp, Artificial Sequence
  synthetic
cggtcgcgtcattttctc

SEQ ID NO: 8 - DNA, 22 bp, Artificial Sequence
  synthetic
gggttatcttcacacaccatga

(19) United States
(12) Patent Application Publication
(10) Pub. No.: US 2015/0190369 A1
(43) Pub. Date: Jul. 9, 2015

(54) QUERCETIN-3-GLUCOSIDE AND USES THEREOF

(71) Applicants: Majambu Mbikay, Ile-Perrot (CA); Francine Sirois, Ottawa (CA); Michel Chretien, Ottawa (CA); Janice Mayne, Ottawa (CA)

(72) Inventors: Majambu Mbikay, Ile-Perrot (CA); Francine Sirois, Ottawa (CA); Michel Chretien, Ottawa (CA); Janice Mayne, Ottawa (CA)

(21) Appl. No.: 14/410,774

(22) PCT Filed: Jun. 28, 2013
(86) PCT No.: PCT/CA2013/050507

§ 371 Date: Dec. 23, 2014

Related U.S. Application Data
(60) Provisional application No. 61/667,736, filed on Jul. 3, 2012.

(51) Int. Cl. A61K 31/366; A61K 31/7048

(52) U.S. Cl. CPC A61K 31/366; A61K 31/7048

(57) ABSTRACT

There is described herein a use of quercetin-3-O-β-D-glucoside (Q3G) for increasing the amount of cell surface low-density lipoprotein receptor (LDLR) on a cell and for reducing the amount of functional proprotein convertase subtilisin/kexin type 9 (PCSK9) secreted by the cell, where the Q3G is formulated for administration to the cell, and where the increase in cell surface LDLR and the decrease in secretion of functional PCSK9 is in comparison to the cell not exposed to Q3G. The use may optionally include the treatment of a statin. There is also described a method of reducing plasma cholesterol levels in a patient in need thereof. The method includes treating the patient with a therapeutically effective amount of Q3G and, optionally, a therapeutically effective amount of a statin.

### FIELD

The present disclosure relates generally to quercetin-3-glucoside. More particularly, the present disclosure relates to quercetin-3-glucoside and its use in reducing plasma cholesterol in a patient.

### BACKGROUND

The body derives its lipids from food and endogenous biosynthesis. Lipids circulate in the body in association with apolipoproteins (apo), forming lipoprotein particles of different densities, depending on their relative content in cholesterol, phospholipids, and triglycerides. Low-density lipoprotein (LDL) is the major cholesterol transporter in humans. The plasma level of LDL-cholesterol (LDL-C) is primarily modulated by the liver. This organ synthesizes cholesterol and packages it into very-LDL (VLDL) particles, which it secretes into the bloodstream. Through its LDL receptor (LDLR), the liver takes up cholesterol from the bloodstream and excretes it into the intestine in bile acids [1]. Excess plasma cholesterol is a risk factor for atherosclerosis and related cardiovascular diseases.

Hepatic clearance of plasma LDL-C is down regulated by proprotein convertase subtilisin/kexin type 9 (PCSK9), the ninth member of the family of proprotein convertases. These subtilases are involved in the post-translational activation or inactivation of secretory proteins by limited endoproteolysis. Human PCSK9 is biosynthesized in the endoplasmic reticulum (ER) as a 692-amino acid preproPCSK9, which, after co-translational removal of a 30-amino acid signal peptide, becomes proPCSK9³¹⁻⁶⁹².

This proPCSK9³¹⁻⁶⁹² zymogen cleaves itself between Gln¹⁵² and Ser¹⁵², generating the PCSK9³¹⁻¹⁵² prosegment and the PCSK9¹⁵³⁻⁶⁹² mature enzyme. The prosegment and the mature enzyme remain attached in a non-covalent, enzymatically inactive complex, which is secreted into the extracellular milieu. The endoproteolytic processing of its zymogen is required for PCSK9 secretion [2]. This has been recently corroborated in humans by the identification of a Gln152His mutation that prevents the cleavage site, causing PCSK9 intracellular retention [3].

Besides endoproteolysis, other post-translational modifications of PCSK9 may include N-glycosylation at Asn⁵³³, sulfation at Tyr³⁸, and phosphorylation at Ser⁴⁷ and Ser⁶⁸⁸ [2,4,5].

The PCSK9/prosegment complex binds to LDLR at the cell surface and, after co-endocytosis, prevents the receptor from returning to the cell surface, rerouting it into lysosomes where it is degraded [6]. The complex is dissociated by a furin-mediated cleavage between Arg²¹⁸ and Gln²¹⁹ in the mature enzyme, producing the ΔNT-PCSK9²¹⁹⁻⁶⁹² devoid of LDLR-degradation activity [4,7]. Thus, hepatic LDLR/PCSK9 expression or activity ratio strongly influences the circulating levels of cholesterol. In humans, hypercholesterolemia has been associated with loss-of-function mutations in the LDLR gene, as well as gain-of-function mutations in the PCSK9 gene [8,9].

High plasma cholesterol levels (i.e. hypercholesterolemia) is a risk factor for atherosclerosis and related cardiovascular diseases. Today, atherosclerosis and related cardiovascular diseases have become global epidemics [10,11]. Statins are the drugs most commonly used to combat them [12]. However, for all their success, statin inhibitors of cholesterol biosynthesis occasionally cause serious side effects, such as myopathy and hepatotoxicity [13], precluding their therapeutic use in a growing number of hypercholesterolemic patients.

Statins reduce intracellular cholesterol biosynthesis by inhibiting 3-hydroxy-3-methylglutaryl coenzyme A reductase (HMGCoAR), the rate-limiting enzyme in cholesterol biosynthesis. This inhibition results in compensatory up-regulation of sterol regulatory element-binding protein 2 (SREBP-2), the transcription factor that drives cholesterol biosynthesis. SREBP-2 activates transcription of both the LDLR and the PCSK9 genes in hepatocytes [14]. Furthermore, therapeutic use of statins in humans is associated with increased plasma levels of PCSK9 [15-17].

The coordinated up-regulation of both the LDLR and PCSK9 genes by statins limits the increase of hepatic LDLR, the efficiency at plasma LDL-C clearance and, therefore, the therapeutic efficacy of the drugs. However, targeted reduction of PCSK9 expression or activity has been shown to potentiate the hypocholesterolemic effect of statins [18-20]. Accordingly, it is believed that PCSK9 inhibitors represent a promising novel class of anti-cholesterol drugs [9,21].

In order to reduce the levels of plasma cholesterol, it is desirable to provide a compound to both increase the level of LDLR and reduce the level of functional, secreted PCSK9 in a patient administered such a compound, since such changes would be expected to increase the cellular uptake of LDL from the blood stream and reduce the levels of plasma cholesterol in the patient.

### SUMMARY

It is an object of the present disclosure to provide the use of quercetin-3-O-β-D-glucoside (Q3G) for increasing the amount of cell surface low-density lipoprotein receptor (LDLR) on a hepatocyte cell and reducing the amount of functional proprotein convertase subtilisin/kexin type 9 (PCSK9) secreted by the hepatocyte cell, where the Q3G is formulated for administration to the hepatocyte cell, and where the increase in cell surface LDLR and the decrease in secretion of functional PCSK9 is in comparison to the hepatocyte cell not exposed to Q3G.

The Q3G may be formulated for administration to provide a concentration of Q3G at the hepatocyte cell, in the extracellular medium, between about 0.1 μM and about 100 μM.

The Q3G may be formulated for administration to a patient having dyslipidemia where the increased amount of cell surface LDLR on the hepatocyte cell and the reduced amount of functional PCSK9 secreted by the hepatocyte cell is for treating metabolic syndrome, or a hypercholesterolemia related-disease or disorder.

The hypercholesterolemia related-disease or disorder may be an obesity-related disease, atherosclerosis, coronary artery disease, stroke, or type 2 diabetes.

The Q3G may be formulated for oral administration.

In another aspect, there is provided the use of quercetin-3-O-β-D-glucoside (Q3G) for reducing the amount of cell surface low-density lipoprotein receptor (LDLR) on a pancreatic beta cell and increasing the amount of functional proprotein convertase subtilisin/kexin type 9 (PCSK9) secreted by the pancreatic beta cell, where the Q3G is formulated for administration to the pancreatic beta cell, and where the decrease in cell surface LDLR and the increase in secretion of functional PCSK9 is in comparison to the pancreatic beta cell not exposed to Q3G.

The Q3G may be formulated for administration to provide a concentration of Q3G at the pancreatic beta cell, in the extracellular medium, between about 4 μM and about 100 μM.

The Q3G may be formulated for administration to a patient having dyslipidemia where the decreased amount of cell surface LDLR on the pancreatic beta cell and the increased amount of functional PCSK9 secreted by the pancreatic beta cell is for reducing cytotoxic effects associated with cholesterol uptake by the pancreatic beta cell.

The hypercholesterolemia related-disease or disorder may be an obesity-related disease, atherosclerosis, coronary artery disease, stroke, or type 2 diabetes.

The Q3G may be formulated for oral administration.

In yet another aspect, there is provided the use of quercetin-3-O-β-D-glucoside (Q3G) in combination with a statin for increasing the amount of cell surface low-density lipoprotein receptor (LDLR) on a hepatocyte cell and reducing the amount of functional proprotein convertase subtilisin/kexin type 9 (PCSK9) secreted by the hepatocyte cell, where the Q3G and the statin are formulated for administration to the hepatocyte cell, where the increase in cell surface LDLR is in comparison to the hepatocyte cell not exposed to either the Q3G or the statin, and where the decrease in secretion of functional PCSK9 is in comparison to the hepatocyte cell exposed to the statin but not exposed to Q3G.

The Q3G may be formulated for administration to provide a concentration of Q3G at the hepatocyte cell, in the extracellular medium, between about 0.1 μM and about 100 μM.

The statin may be simvastatin.

The Q3G and the statin may be formulated for administration to a patient having dyslipidemia where the increased amount of cell surface LDLR on the hepatocyte cell and the reduced amount of functional PCSK9 secreted by the hepatocyte cell is for treating metabolic syndrome, or a hypercholesterolemia related-disease or disorder.

The hypercholesterolemia related-disease or disorder may be an obesity-related disease, atherosclerosis, coronary artery disease, stroke, or type 2 diabetes.

The Q3G may be formulated for oral administration.

In still another aspect, there is provided a composition comprising quercetin-3-O-β-D-glucoside (Q3G) and a statin, the composition for increasing the amount of cell surface low-density lipoprotein receptor (LDLR) on a hepatocyte cell and reducing the amount of functional proprotein convertase subtilisin/kexin type 9 (PCSK9) secreted by the hepatocyte cell, where the increase in cell surface LDLR is in comparison to the hepatocyte cell not exposed to either the Q3G or the statin, and where the decrease in secretion of functional PCSK9 is in comparison to the hepatocyte cell exposed to the statin but not exposed to Q3G.

The statin may be simvastatin.

In yet another aspect, there is provided a composition comprising quercetin-3-O-β-D-glucoside (Q3G) and a statin, the composition for: increasing the amount of cell surface low-density lipoprotein receptor (LDLR) on a hepatocyte cell and reducing the amount of functional proprotein convertase subtilisin/kexin type 9 (PCSK9) secreted by the hepatocyte cell, where the increase in cell surface LDLR is in comparison to the hepatocyte cell not exposed to either the Q3G or the statin, and where the decrease in secretion of functional PCSK9 is in comparison to the hepatocyte cell exposed to the statin but not exposed to Q3G; and reducing the amount of cell surface low-density lipoprotein receptor (LDLR) on a pancreatic beta cell and increasing the amount of functional proprotein convertase subtilisin/kexin type 9 (PCSK9) secreted by the pancreatic beta cell, where the decrease in cell surface LDLR is in comparison to the pancreatic beta cell not exposed to either the Q3G or the statin, and where the increase in secretion of functional PCSK9 is in comparison to the pancreatic cell exposed to the statin but not exposed to Q3G.

In a further aspect, there is provided a method of increasing the amount of cell surface low-density lipoprotein receptor (LDLR) on a hepatocyte cell and reducing the amount of functional proprotein convertase subtilisin/kexin type 9 (PCSK9) secreted by the hepatocyte cell, the method including: treating the hepatocyte cell with an effective concentration of quercetin-3-O-β-D-glucoside (Q3G) the increase in cell surface LDLR and the decrease in secretion of functional PCSK9 being in comparison to the hepatocyte cell prior to treatment with the Q3G.

The effective concentration of Q3G at the hepatocyte cell, in the extracellular medium, may be between about 0.1 μM and about 100 μM.

In a still further aspect, there is provided a method of not substantially changing, or of decreasing the amount of cell surface low-density lipoprotein receptor (LDLR) on a pancreatic beta cell, and increasing the amount of functional proprotein convertase subtilisin/kexin type 9 (PCSK9) secreted by the pancreatic beta cell, the method including: treating the pancreatic beta cell with an effective concentration of quercetin-3-O-β-D-glucoside (Q3G) the increase in cell surface LDLR and the decrease or lack of substantial change in secretion of functional PCSK9 being in comparison to the pancreatic beta cell prior to treatment with the Q3G.

The effective concentration of Q3G at the pancreatic beta cell, in the extracellular medium, may be between about 4 μM and about 100 μM.

In another aspect, there is provided a method of increasing the amount of cell surface low-density lipoprotein receptor (LDLR) on a hepatocyte cell and reducing the amount of functional proprotein convertase subtilisin/kexin type 9 (PCSK9) secreted by the hepatocyte cell, the method including: treating the hepatocyte cell with an effective amount of quercetin-3-O-β-D-glucoside (Q3G) and a statin, the increase in cell surface LDLR being in comparison to the hepatocyte cell not exposed to either the Q3G or the statin, and the decrease in secretion of functional PCSK9 being in comparison to the hepatocyte cell exposed to the statin but not exposed to Q3G.

In a still further aspect, there is provided a method of reducing plasma cholesterol levels in a patient in need thereof, the method including: administering to the patient a therapeutically effective amount of quercetin-3-O-β-D-glucoside (Q3G) to increase the amount of cell surface low-density lipoprotein receptor (LDLR) on a hepatocyte cell and to reduce the amount of functional proprotein convertase subtilisin/kexin type 9 (PCSK9) secreted by the hepatocyte cell, thereby increasing rate of cellular uptake of exogenous LDL from the plasma of the patient and reducing the plasma cholesterol levels in the patient, the increase in cell surface LDLR and the decrease in secretion of functional PCSK9 being in comparison to the hepatocyte cell prior to exposure to the Q3G.

Administration of the Q3G may increase the amount of functional PCSK9 secreted by a pancreatic beta cell and decrease the amount of cell surface LDLR on the pancreatic beta cell, the decrease or lack of substantial change in cell surface LDLR and the increase in secretion of functional PCSK9 being in comparison to the pancreatic beta cell prior to exposure to the Q3G.

The reduction of plasma cholesterol may result in the treatment or prevention of metabolic syndrome, or a hypercholesterolemia related-disease or disorder.

The hypercholesterolemia related-disease or disorder may be an obesity-related disease, atherosclerosis, coronary artery disease, stroke, or type 2 diabetes.

The Q3G may be orally administered to the patient.

In still a further aspect, there is provided a method of reducing plasma cholesterol levels in a patient in need thereof, the method including: administering to the patient a therapeutically effective amount of quercetin-3-O-β-D-glucoside (Q3G) and a therapeutically effective amount of a statin; where treatment of the patient with the Q3G and the statin increases the amount of cell surface low-density lipoprotein receptor (LDLR) on a hepatocyte cell when compared to the hepatocyte cell not exposed to either the Q3G or the statin, and reduces the amount of functional proprotein convertase subtilisin/kexin type 9 (PCSK9) secreted by the hepatocyte cell in comparison to the hepatocyte cell exposed to the statin but not exposed to Q3G, the increased amount of hepatocyte cell surface LDLR and reduced amount of functional PCSK9 secreted by the hepatocyte cell resulting in an increased rate of cellular uptake of exogenous LDL from the plasma of the patient and a reduced level of plasma cholesterol in the patient.

The treatment of the patient with the Q3G may increase the amount of functional PCSK9 secreted by a pancreatic beta cell and decrease or not substantially change the amount of cell surface LDLR on the pancreatic beta cell, the decrease or lack of substantial change in cell surface LDLR and the increase in secretion of functional PCSK9 being in comparison to the pancreatic beta cell prior to exposure to the Q3G.

The reduction of plasma cholesterol may result in the treatment or prevention of metabolic syndrome, or a hypercholesterolemia related-disease or disorder.

The hypercholesterolemia related-disease or disorder may be an obesity-related disease, atherosclerosis, coronary artery disease, stroke, or type 2 diabetes.

The Q3G may be orally administered to the patient.

Other aspects and features of the present disclosure will become apparent to those ordinarily skilled in the art upon review of the following description of specific embodiments in conjunction with the accompanying figures.

### BRIEF DESCRIPTION OF THE DRAWINGS

Embodiments of the present disclosure will now be described, by way of example only, with reference to the attached Figures.

FIG. 1 is a graph illustrating dose dependent reduction of PCSK9 with an aqueous extract of M. oleifera leaves.

FIG. 2 is an illustration of the chemical structure of quercetin-3-O-β-D-glucoside (Q3G).

FIG. 3 is a graph illustrating hepatocyte nuclear factor la (HNF-1α) expression in cells on exposure to Q3G. Cells were incubated for 24 h in medium containing the indicated concentrations of Q3G. Cells lysates were analyzed by semi-quantitative immunoblotting for the levels of HNF-1α.

FIG. 4 is a graph illustrating the spectrometry of PCSK9-Q3G interaction.

FIGS. 5A and 5B are graphs illustrating LDLR mRNA and protein levels in cells exposed to Q3G. Cells were incubated for 24 h in medium containing the indicated concentrations of Q3G. FIG. 5A illustrates the results for quantitative RT-PCR for LDLR levels. FIG. 5B illustrates semi-quantitative immunoblotting for LDLR. Values are the means of triplicate experiments±and standard errors of means (SEM). Different letters above bars mean significant difference (P<0.05).

FIGS. 6A, 6B(a) and 6B(b) are graphs illustrating PCSK9 mRNA and protein levels for cells exposed to Q3G. Cells were incubated for 24 h in medium containing the indicated concentrations of Q3G. FIG. 6A illustrates the results for quantitative RT-PCR for PCSK9 mRNA levels. FIG. 6B(a) illustrates the results for semi-quantitative immunoblotting for cellular PCSK9. FIG. 6B(b) illustrates the results for ELISA for secreted PCSK9 in conditioned media. Values are means of triplicate experiments±SEM. Different letters above bars mean significant difference (P<0.05).

FIG. 7 is a graph illustrating LDLR levels for hepatocyte cells exposed to various concentrations of Q3G. Hepatocyte cells were incubated in medium containing the indicated Q3G concentrations for 24 h. LDLR was analyzed by immunoblotting and its content normalized for that of transferin receptor (TfR). Values are the means of 3 separate experiments±SEM.

FIG. 8 shows graphs illustrating a time course of Q3G-induced LDLR and PCSK9 cellular levels. Cells were incubated in medium containing 2 μM Q3G for the indicated length of time. Cells lysates were analyzed by semi-quantitative immunoblotting for the levels of LDLR and PCSK9. Values are the means of triplicate experiments±SEM.

FIGS. 9A and 9B are graphs illustrating the proSREBPs-2 mRNA and SREBP-2-related protein levels for cells exposed to Q3G. Cells were incubated for 24 h in medium 5 μM Q3G. FIG. 9A illustrates the results for quantitative RT-PCR for proSREBPs-2 mRNA levels. Values are the means of triplicate experiments±SEM. FIG. 9B illustrates the results for semi-quantitative immunoblotting for cellular SREBP-2-related protein. Mat/Prec values, the averages of two experiments, represent density ratios of the 65-kDa SREBP over the 158-kDa proSREBPs after normalization for β-actin.

FIGS. 10A and 10B are phosphor-images of PCSK9-related proteins in cell lysates and in conditioned media, respectively. FIG. 10C is a graph illustrating the quantified proteins from FIGS. 10A and 10B. Cells were pre-incubated for 24 h in medium 5 μM Q3G. After metabolic labeling with radioactive amino acids, labeled proteins were chased in Q3G-free non-radioactive medium, for varying lengths of time. PCSK9-related proteins were immunoprecipitated, fractionated by SDS-PAGE, and quantified by phosphorimaging. FIG. 10A shows the images for PCSK9-related proteins in cell lysates. FIG. 10B shows the images for PCSK9-related proteins in conditioned media. FIG. 10C is a graph showing the percent of medium PCSK9 signals over to the total of intracellular and extracellular PCSK9 signals.

FIGS. 11A-C are graphs illustrating reduction of statin-induced PCSK9 secretion by Q3G. Huh7 cells were incubated for 24 h in culture medium containing simvastatin (SMV: 0, 0.2, or 1 mM), without or with 5 μM Q3G. The levels LDLR and PCSK9 in cell extracts were evaluated by immunoblotting. The levels of PCSK9 in spent media were determined by ELISA. Different letters above bars mean significant difference (P<0.05)

FIG. 12 shows a flow cytometry plot and confocal microscopy image of cells stained to detect LDLR. Cells were pre-treated or not with 5 μM Q5G. They were then stained for LDLR by indirect immunofluorescence and analyzed by immunofluorescence flow cytometry. The experiment was conducted in triplicates. The figure shows mean fluorescence±SEM. ***, P<0.001 by Student's t test. The image is a confocal microscopy image of cell surface LDLR stained for LDLR by indirect immunofluorescence and counterstained with propidium iodide to visualize the nuclei.

FIG. 13 is a graph illustrating the increase in LDL secretion in cells exposed to Q3G. Cells were pre-treated or not with 5 μM Q5G. They were then incubated with fluorescent bodipy-LDL for up to 30 min. Intracellular fluorescence was measured by fluorescence spectrometry. Values represents means of 6 replicates±SEM. ***, P<0.005; **, P<0.01 by Student t test.

FIG. 14 is a graph illustrating the effect of exposure to Q3G on the levels of PCSK9, LDLR, ABCA1 and ABCG1 mRNA in MIN6 β-cells. MIN6 cells were incubated for 24 h in the presence of the specified concentration of Q3G. Total RNA was extracted and analyzed for the levels of mRNA of the specified protein, followed by normalization for the levels of TBP mRNA. The values are plotted taking the values of each molecule at 0 μM Q3G as 1.

FIG. 15 a graph illustrating the effect of exposure to Q3G on PCSK9 secretion in MIN6 β-cells. MIN6 cells were incubated for 24 h in the presence of the specified concentration of Q3G. Media were collected and assayed by ELISA for PCSK9 content.

FIG. 16 shows graphs illustrating the relative levels of the cellular content of lipid modulatory proteins (PCSK9, LDLR, ABCA1 and ABCG1 proteins) in MIN6 β-cells that were untreated or treated with 16 μM Q3G. The corresponding photographs of the immunoblotting results are also shown. MIN6 cells were incubated for 24 h in the presence of 16 μM Q3G. Cell lysates were analyzed by semi-quantitative immunoblotting using different antibodies successively.

FIGS. 17A and 17B are graphs illustrating the effect of exposure to Q3G on insulin and PCSK9 secretion in MIN6 β-cells. MIN6 cells were incubated for 24 h in medium with or without Q3G. Medium containing 3 mM Glucose (low glucose) with or without Q3G was substituted and incubation resumed for 6 h. Fresh low glucose medium with or without Q3G was substituted and supplemented or not with additional glucose to the final concentration of 18 μM. After 30 min of incubation, media were collected and assayed by ELISA for insulin and PCSK9

### DETAILED DESCRIPTION

Generally, the present disclosure provides a compound that both increases the amount of cell-surface LDL-receptor on a hepatocyte cell and reduces the amount of functional proprotein convertase subtilisin/kexin type 9 (PCSK9) secreted by the hepatocyte cell. The compound is quercetin-3-O-β-D-glucoside (Q3G). For example, Q3G reduces the amount of PCSK9 secreted by the hepatocyte cell, increasing the half-life of cell-surface LDL-receptor on the hepatocyte cell, and stimulating cholesterol clearance from the blood.

The Q3G also decreases the amount, or does not substantially change the amount, of cell-surface LDL-receptor on a pancreatic beta cell, and increases the amount of functional proprotein convertase subtilisin/kexin type 9 (PCSK9) secreted by the pancreatic beta cell. In one example, Q3G increases the amount of PCSK9 secreted by the pancreatic beta cell, reducing the half-life of cell-surface LDL-receptor on the pancreatic beta cell, and protecting the beta cell from lipotoxic effects of excessive LDL-cholesterol uptake mediated by the LDL-receptor.

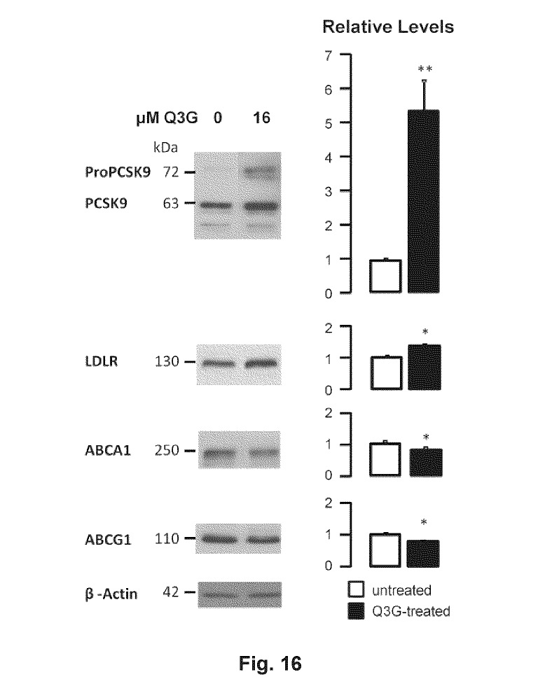

In the context of the present disclosure, it would be understood that “not substantially changing the amount of cell-surface LDL-receptor on a pancreatic beta cell” would correspond to an increase or a decrease of no more than 50% in comparison to the amount of cell-surface LDL-receptor on the pancreatic beta cell which has not been exposed to Q3G. For example, Example 7 and FIG. 16 illustrate that the amount of cell-surface LDL-receptor on MIN6 β-cells that have been exposed to 16 μM Q3G is approximately 1.4 times greater than the amount of cell-surface LDL-receptor on untreated MIN6 β-cells. Treatment with Q3G would be considered, in the context of the present disclosure, to not substantially change the amount of the cell-surface LDL-receptor.

Q3G may be considered a PCSK9 antagonist in hepatocyte cells, and a PCSK9 agonist in pancreatic beta cells.

An increase in the amount of cell-surface LDL-receptor and reduction in the amount of functional, secreted PCSK9 in hepatocyte cells may reduce plasma cholesterol levels in a patient treated with the compound due to accelerated cellular uptake of exogenous LDL. Increasing the amount of functional, secreted PCSK9 cells, while at the same time reducing or not substantially changing the amount of cell-surface LDL-receptor on pancreatic beta cells may reduce the likelihood of insulin insufficiency, impaired glucose-stimulated insulin secretion, or both.

Reduction in plasma cholesterol levels may be beneficial in treating metabolic syndrome, or hypercholesterolemia related-diseases or disorders. Examples of diseases or disorders which may be treated through a reduction in plasma cholesterol levels include: obesity-related diseases, atherosclerosis, coronary artery disease, stroke, and type 2 diabetes.

Other diseases or disorders which may be treated through a reduction in plasma cholesterol include: Alzheimer's disease, cancer and infectious diseases such as malaria and human immunodeficiency virus (HIV), since cholesterol and cholesterol-rich lipid rafts have been implicated in these diseases. It is believed that reduction of the level of circulating cholesterol may interfere with the pathophysiology of these diseases or disorders. Generally, any disease requiring high cholesterol for its progression may be targeted for treatment with a compound that both increases the amount of LDL-receptor on hepatocyte cells and reduces the amount of functional, secreted PCSK9 secreted by the hepatocyte cells.

The amount of cell-surface LDL-receptor in the liver, 70-85% by mass of which is made up of hepatocyte cells, may be indirectly measured by measuring clearance of plasma LDL levels since liver LDL-receptors are responsible for about 90% of the clearance of plasma LDL. Plasma LDL may be measured by standard techniques. Secreted PCSK9 may be determined using an ELISA assay, such as in commercially available assays from MBL International or R&D Systems.

As some plants have been shown to display anti-cholesterolemic properties [22], these plants were analyzed to determine if they contained compounds that increased the amount of cell-surface LDL-receptor, reduced the amount of functional, secreted PCSK9, or both increased the amount of cell-surface LDL-receptor and reduced the amount of functional, secreted PCSK9. Specifically, Moringa oleifera, Lam (M. oleifera), a perennial plant of the tropics, whose leaves have been shown to exhibit anti-dyslipidemic properties in experimental animals and in humans [23-27] was analyzed.

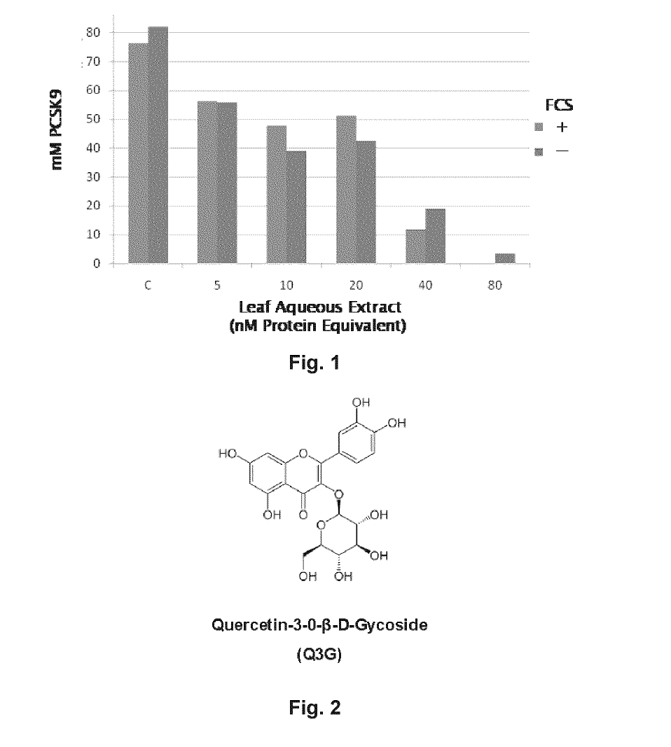

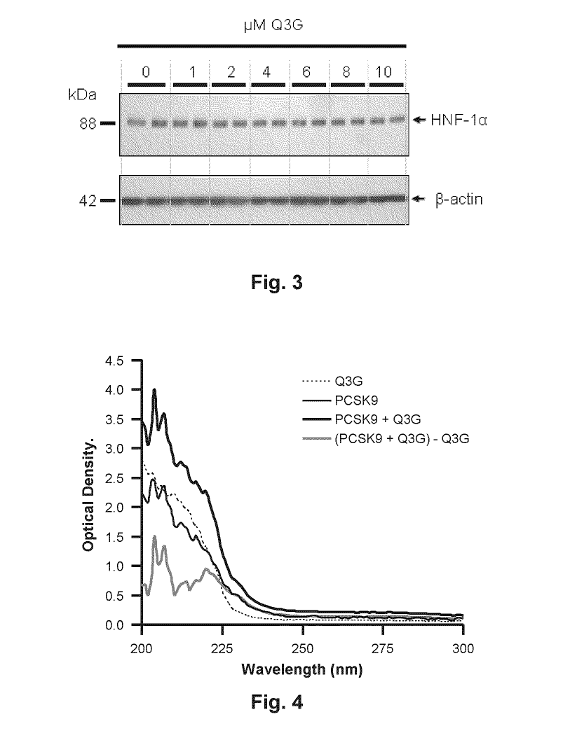

It was observed that exposure of Huh7 human hepatocytes in culture to an aqueous extract of M. oleifera leaves significantly reduced the amounts of PCSK9 secreted in the culture medium, in a concentration dependent manner, as illustrated in FIG. 1. HuH7 cells were incubated for 24 h in medium containing (+) or not (−) 10% fetal calf serum (FCS), supplemented or not (C) with an aqueous extract of Moringa oleifera (Mo) leaf dried leaf powder. Media were collected and PCSK9 levels therein were determined by ELISA. The dried Mo leaf powder originated from Burundi. It was suspended at 10% in sterile distilled water, boiled for 5 min and filtered under vacuum. The protein concentration in the filtrate was determined using the Bio-Rad dye method. The figure represents means of 3 separate experiments.

The bioflavonoid quercetin was identified as a candidate compound for the observed anti-PCSK9 activity of the plant. Quercetin is found in amounts as high as 1 mg/g of Moringa oleifera leaf powder [28], predominantly as quercetin-3-O-β-D-glucoside (Q3G) [29,30] (FIG. 1). This flavonoid has been previously shown to reduce diet-induced hyperlipidemia and atherosclerosis in rabbits [31,32] and to attenuate the metabolic syndrome of obese Zucker rats [33]. However, until this point, no metabolic basis for these results has been determined.

It was further observed that exposure of MIN6 β-cells (a mouse insulinoma cell line) in culture to Q3G stimulates PCSK9 expression and secretion, without affecting glucose-stimulated insulin secretion (GSIS).

Based on the results discussed herein, it has now been established that quercetin-3-O-β-D-glucoside: increases the amount of cell-surface LDLR and inhibits PCSK9 secretion in hepatocytes. It has also been established that the Q3G stimulates PCSK9 secretion while at the same time reduces or does not substantially change the cell-surface level of LDL-receptor in pancreatic beta cells.

Accordingly, the present disclosure provides a method of increasing the amount of cell-surface LDL-receptor on hepatocyte cells and reducing the amount of functional, secreted PCSK9 secreted by the hepatocyte cells. For example, Q3G reduces the amount of PCSK9 secreted by the hepatocyte cell, increasing the half-life of cell-surface LDL-receptor on the hepatocyte cell, and stimulating cholesterol clearance from the blood.

The present disclosure also provides a method of not substantially changing or decreasing the amount of cell-surface LDL-receptor on a pancreatic beta cell while at the same time increasing the amount of functional proprotein convertase subtilisin/kexin type 9 (PCSK9) secreted by the pancreatic beta cell. For example, Q3G increases the amount of PCSK9 secreted by the pancreatic beta cell, reducing the half-life of cell-surface LDL-receptor on the pancreatic beta cell, and protecting the beta cell from lipotoxic effects of excessive LDL-cholesterol uptake mediated by LDL-receptor.

Such an increase in the amount of cell-surface LDL-receptor on the hepatocytes and reduction in the amount of functional, secreted PCSK9 secreted by the hepatocytes is expected to reduce plasma cholesterol levels in a patient treated with quercetin-3-O-β-D-glucoside due to accelerated cellular uptake of exogenous LDL. Reduction in plasma cholesterol levels are expected to be beneficial in treating metabolic syndrome, or hypercholesterolemia related-diseases or disorders. Examples of diseases or disorders which are expected to be treated through a reduction in plasma cholesterol levels include: atherosclerosis, coronary artery disease, stroke, and type 2 diabetes.

The treatment with Q3G may be especially beneficial to the cardiovascular system when the treatment results in: hepatocytes with increased amounts of cell-surface LDL-receptor; and pancreatic beta cells with increased amount of secreted PCSK9 and with substantially unchanged or reduced amounts of LDL-receptor. Increasing the amount of secreted PCSK9 in pancreatic beta cells, while reducing or leaving the amount of LDL-receptor substantially unchanged, protects the pancreatic beta cells from lipotoxicity resulting from excessive LDL-cholesterol uptake mediated by the LDL-receptor, and therefore helps maintain glucose homeostasis.

Q3G may be administered orally, for example in an oral dose between 150 mg and 1 g. It is believed that oral administration of Q3G will result in an increase in the amount of cell-surface LDL-receptor on hepatocyte cells and a reduction in the amount of functional, secreted PCSK9 secreted by the hepatocyte cells since i) Moringa leaf powder taken orally can effectively reduce cholesterol in animal; ii) Q3G is the predominant form of quercetin in Moringa leaf powder; and iii) Q3G can be taken up by the intestine and its derivatives (sulfated, methylated or glucuronylated) are found in the blood. Q3G may also be administered parenterally (intravenously). Q3Q has been administered intravenously to treat hypertension, as discussed by M. Russo et al. in Biochemical Pharmacology 83 (2012) 6-15.

The results discussed herein indicate that in vitro exposure of Huh7 hepatocytes with Q3G (i) stimulates proSREBP-2 proteolytic activation, (ii) increases the levels of LDLR mRNA and protein, (iii) increases the cell surface density of LDLR, (iv) reduces the cellular levels of PCSK9 mRNA, (v) reduces PCSK9 accumulation in the culture medium and (vi) accelerates cellular uptake of exogenous LDL.

Although the examples disclosed herein were performed at low micromolar concentrations (i.e. concentrations between 2 μM and 50 μM), it is expected that Q3G may be administered at an in vivo concentration of about 0.1 μM to about 100 μM and still result in, in hepatocytes: (i) stimulation of proSREBP-2 proteolytic activation, (ii) increased levels of LDLR mRNA and/or protein, (iii) increased cell surface density of LDLR, (iv) reduced levels of PCSK9 mRNA, (v) reduced PCSK9 accumulation in the culture medium, (vi) accelerated cellular uptake of exogenous LDL, or (vii) any combination thereof. In certain examples, a therapeutically effective dose is a dose administered such that the recipient's plasma level of Q3G is in the range of 0.5 to 5 μM. This may be achieved, for example, through the oral administration of about 2 mg of Q3G/kg of body weight. See, for example, K. Murota et al. Achives of Biochemistry and Biophysics 501 (2010) 91-97.

In view of the present disclosure, it is expected that in vivo exposure of hepatocytes to Q3G would similarly: (a) increases the cell surface density of LDL-receptor on the hepatocytes and (b) reduce the level of functional, secreted PCSK9 secreted by the hepatocytes. This increased cell surface density of LDLR and reduced levels of functional, secreted PCSK9 would similarly be expected to accelerate cellular uptake of plasma LDL and lead to a reduction in plasma cholesterol levels, though the reduction in plasma cholesterol levels is due, in vivo, to hepatocytes and the impact of extrahepatic tissues in plasma cholesterol levels is overshadowed by the impact of the hepatocytes. Such a reduction in plasma cholesterol levels is expected to be beneficial in treating metabolic syndrome, or hypercholesterolemia related-diseases or disorders. Examples of diseases or disorders which may be treated through a reduction in plasma cholesterol levels include: obesity-related diseases, atherosclerosis, coronary artery disease, stroke, and type 2 diabetes.

Without wishing to be bound by theory, the in vitro accelerated uptake of exogenous LDL is believed to at least partially be due to a higher density of LDLR at the cell surface of the hepatocytes, following stimulated expression of its gene by SREBP-2. However, the 2× increase of LDLR mRNA could not, alone, account for the 4× increase in the LDLR level. It is also believed that the protein half-life was also increased, since the level of secreted PCSK9 decreased. Indeed, although an intracellular LDLR-degrading activity has been suggested for PCSK9 [39], the remarkable hypocholesterolemic efficacy of parenteral therapy using anti-PCSK9 antibodies [40,41] is evidence that the primary mechanism of action of PCSK9 involves its prior secretion and its subsequent binding to the LDL receptor at the cell surface. The attenuation of LDLR increase when Huh7 cells were exposed to Q3G above 2-digit micromolar concentrations may be due to feedback repression of the LDLR gene following the intracellular accumulation of cholesterol caused by the flavonoid.

Without wishing to be bound by theory, the reduction of cellular levels of PCSK9 mRNA in hepatocytes following treatment with Q3G is believed to result from invalidation of co-activators of the PCSK9 gene promoter, induction of repressors of this promoter, increased instability of the transcript, or a combination thereof. Berberine (BBR) which, like Q3G, is a plant-derived hypocholesterolemic compound, reduces PCSK9 gene transcription by inducing decreased expression of hepatocyte nuclear factor la (HNF-1α). This factor cooperates with SREBP-2 to activate the PCSK9 promoter. In its absence, the promoter activity is reduced [42]. Unlike BBR, Q3G does not change the level of HNF-1α (FIG. 3), suggesting that Q3G prevents PCSK9 gene activation by SREBP-2 through a different mechanism.

The data discussed herein indicate that chronic exposure of Huh7 cells to Q3G reduces PCSK9 accumulation in the culture medium by delaying its transit through the secretory pathway. The delay appears not to be caused by impaired proteolytic processing of its precursor. Quercetin is known to bind, covalently in some cases, to selected cellular proteins [43-45]. Without wishing to be bound by theory, the spectroscopy data discussed herein suggest that Q3G can bind to recombinant human PCSK9 in vitro, as illustrated in FIG. 4. Purified recombinant PCSK9 (5μM) was mixed with or without equimolar amount of Q3G in phosphate-buffered saline. After a 5-min incubation, the UV spectrum of the mix was taken. The changes of PCSK9 optical density and spectral profile upon Q3G addition suggest interaction between these two molecules. It is believed that such a binding may alter PCSK9 conformation and/or retard its navigation through the secretory pathway, and, ultimately, diminish its LDL-degrading activity.

PCSK9 has been recently shown to interact with Apo B, protecting it from autophagic degradation [46]. Quercetin aglycone, at 5-30 μM, has been shown to inhibit Apo B secretion by intestinal Caco-2 cells. The inhibition was selective since there was no difference between treated and untreated cells in the overall amount of secreted proteins after a 2-h metabolic pulse-labeled with radioactive amino acids. In this case, inhibition of Apo B secretion appeared to be caused by reduced packaging of triacylglyceride to the protein [47]. Interference with normal intermolecular interactions is one of possible mechanisms of Q3G-induced delay of PCSK9 secretion.

Inhibition of PCSK9 secretion or an increase in LDLR level in Huh7 cells exposed to quercetin aglycone was not observed at the concentrations of Q3G discussed herein. Another recent study has reported LDLR up-regulation in HepG2 hepatocytes with 75 μM of the non-glycosylated form of quercetin [48]. Without wishing to be bound by theory, it is believed that the greater effectiveness of the glycosylated form of quercetin may be due to its ability to enter into cells more efficiently, to interact more strongly with functional proteins at the cell surface or within the cell, or a combination thereof.

In pigs and dogs fed a meal supplemented with either quercetin aglycone, Q3G, or quercetin-3-O-glucorhamnoside (rutin), quercetin bioavailability was significantly greater with Q3G as a supplement than with the other two forms of quercetin [49,50]. Intestinal Na-dependent glucose transporter 1 (SGLT1) appears to mediate this preferential uptake [51]. Yet quercetin aglycone has been shown to penetrate, passively or actively, inside a variety of other cell types [52], including HepG2 hepatocytes, where it elicited significant changes in gene expression [53].

Statins induce expression of LDLR and PCSK9. However, unlike Q3G, statins do not reduce PCSK9 secretion. Administration of Q3G to a patient may be used to reduce the level of functional, secreted PCSK9 secreted by hepatocyte cells which is stimulated by the administration of an inhibitor of HMGCoA reductase, for example a statin such as simvastatin, to the patient. Example 4 discusses the treatment of hepatocytes with Q3G and/or simvastatin. The results suggest that simvastatin and Q3G stimulated LDLR expression through similar mechanism; but that Q3G possesses, in addition, distinct anti-PCSK9 production/secretion properties. It is expected that Q3G could similarly be used to reduce the stimulated level of functional, secreted PCSK9 in a patient administered a statin other than simvastatin. The level of secreted, plasma PCSK9 in a patient may be measured using commercially available ELISA kits.

### EXAMPLES

Materials

Huh7 human liver cells and the rabbit anti-human PCSK9 antibody for immunoblotting were obtained from Dr. Nabil G Seidah. The rabbit anti-human PCSK9 antibody for immunoprecipitation was produced in house. The following antibodies were from commercial sources: anti-LDLR (RD Systems), anti-β-actin and simvastatin (Sigma), anti-SREBP-2 (Santa Cruz), Horseradish peroxidase (HRP)-conjugated antirabbit or mouse immunoglobulins (Ig) (GE HealthCare) or anti-goat Ig (Santa Cruz). The chemiluminescence revelation kit was from PerkinElmer; the PCSK9 ELISA kit from Circulex or RD Systems; the RNeasy extraction kit from Qiagen. Superscript II RNase H-Reverse Transcriptase, bodipy-LDL, non-conjugated LDL; lipoprotein-depleted serum (LPDS), and Alexa Fluor 488™ were from Invitrogen. The FastStart TaqMan ProbeMaster-Rox master mix, primer pairs, and Universal Probe Library (UPL) fluorescent probes and Protease Inhibitor Cocktail (PIC) were from Roche, and Amplify fluor solution from Amersham Biosciences. Q3G was obtained from Sigma; goat anti-mouse LDLR from Cederlane; anti-β-actin monoclonal primary anti-body and horseradish (HRP)-conjugated donkey anti-goat IgG from Santa Cruz; HRP-conjugated sheep anti-mouse IgG from GE HealthCare; ELISA kit for mouse PCSK9 and mouse insulin from R & D Systems, and Crystal Chem, respectively; the protease inhibitor cocktail (PIC), the FastStart TaqMan ProbeMaster-Rox master mix, primer pairs and fluorescent probes from Roche; the RNA extraction kit from Qiagen, Super-script II RNase H-Reverse Transcriptase from Invitro-gen, the Western Lightning Chemiluminescence Reagent Plus a chemiluminescence-based revelation kit from Perkin-Elmer.

Cell Culture and Lysis

At passage, Huh7 cells were routinely seeded at sub-confluence (˜10⁶ cells/10-cm dish) in Dulbecco's modified Eagle's medium (DMEM) containing 10% fetal bovine serum (FBS) or LPDS (for experiments) and 50 μg/ml gentamycin. They were incubated overnight at 37° C., in a humidified 5% CO₂-95% air atmosphere. Cells were treated or not with Q3G at defined concentrations and for defined lengths of time. Media were collected and centrifuged at 200 g for 5 min to sediment suspended cells; supernatants were collected and supplemented with 0.33 volumes of a 3×-concentrated PIC. Cell monolayers were rinsed with ice-cold phosphate-buffered saline (PBS); they were overlaid with 0.5 of the RIPA lysis buffer (50 mM Tris-HCl, pH 8, 150 mM NaCl, 1% NP-40, 0.5% Na-deoxycholate and 0.1% SDS) supplemented with 1× PIC. After 20 min in an ice bath, the lysates were centrifuged at 14,000 g and 4° C. for 20 min, and supernatants were collected. Conditioned media and lysates were stored at −20° C. until analysis.

Mouse insulinoma MIN6 cells were cultured in a 5% CO₂-95% air atmosphere at 37° C. in DMEM medium containing 10% heat-inactivated fetal bovine serum, 1 mM Na-pyruvate, 2 mM L-glutamine, 25 mM D-glucose, and 28 μM β-mercaptoethanol. Q3G at a specific final concentration was supplemented to the culture medium and incubation was conducted for a selected length of time. Media were collected, spun at 600 g to sediment suspended cells, supplemented with 0.5 volumes of 3×RIPA-PIC (1×: 50 mM Tris-HCl, pH 8, 150 mM NaCl, 1% NP-40, 0.5% Na-deoxycholate and 0.1% SDS and PIC). Cells were lysed in 1×RIPA-PIC for immuno-blotting, or in RNA extraction buffer for qRT-PCR.

Metabolic Labeling

For metabolic labeling, Huh7 cells were seeded in a 12-well plate 8×10⁵ cells/well in 1.5 ml/well of complete medium and incubated overnight. After a rinse with Dulbecco's PBS (PBS-D), cell monolayers were overlaid with 1.5 ml of DMEM/10% LPDS without or with 5 μM Q3G, and were incubated for 24 h. Fresh serum-free medium (SFM, 1.5 ml) was substituted, and cells were allowed to incubate for 30 min to reduce endogenous Met and Cys. The medium was removed and replaced with fresh SFM (0.75 ml/well) containing 300 μCi/ml³⁵S-Met/Cys, and cells were incubated at 37° C. for 20 min to label de novo biosynthesized proteins (pulse-labeling). The radioactive medium was replaced with DMEM/0.5% LPDS containing 10 mM non-radioactive Met/Cys and cells were incubated at 37° C. for 0, 15, 30, 60, 90 and 120 min (chase). Conditioned media and cell lysates were processed as described above.

Flow Cytometry

Cells were seeded at 4×10⁴ cell/10-cm dish in 3 ml of completed medium and incubated overnight. LPDS medium containing or not 5 μM Q3G was substituted and incubation resumed for 24 h. Subsequent steps were conducted with ice-cold solutions and at 4° C. Cell monolayers were rinsed with PBS, overlaid with PBS containing rabbit anti-LDLR antibody for 1 h, then with PBS containing Alexa Fluor-488-conjugated goat anti-rabbit Ig antibody for another 1 h. After a PBS rinse, the cells were overlaid with Versene, suspended in DMEM and analyzed in Benson-Dickenson XL flow cytometer at 492 nm and 520 nm excitation and emission wavelengths, respectively. Cell autofluorescence and non-specific fluorescence were assessed using cells not treated with the secondary and the primary antibody, respectively.

LDL Uptake Assay

Huh7 cells were seeded in 96-well black-bottom plates at 4×10⁴ cells/well in 0.1 ml complete medium and allowed to attach by overnight incubation at 37° C. They were rinsed with PBS-D, overlaid with 0.1 ml of DMEM/10% LPDS and incubated at 37° C. for 24 h. After a PBS-D rinse, they were overlaid with 0.1 ml DMEM/0.5% LPDS containing or not 5 μM Q3G and incubated 37° C. for 24 h. To assay for LDL uptake ability, cells were rinsed, first with pre-warmed (37° C.) PBS-D, then with pre-warmed DMEM/0.5% LPDS. They were overlaid with 75 μl of the latter medium containing 20 mg/ml bodipy-LDL, and then incubated at 37° C. for 15 min or 30 min to allow LDLR-mediated endocytosis of the fluorescent lipoprotein. The process was stopped by substituting ice cold DMEM/0.5% LPDS. After 3 rinses with 0.2 ml of ice-cold PBS-D, the cells were fixed with 0.1 ml of isopropanol for 20 min, in the dark and with gentle shaking. Intracellular fluorescence was measured in a SpectraMax Gemini XS fluorescence plate reader (Molecular Devices) at the excitation and emission wavelengths of 485 and 535 nm, respectively. Non-specific fluorescence was measured by incubating cells in medium containing bodipy-LDL (20 μg/ml) and a 12.5× excess of non-fluorescent LDL (250 μg/ml).

RT-qPCR

Total RNA was extracted using the Qiagen RNeasy extraction kit. It was reverse transcribed into cDNA using random hexameric primers and the Superscript II RNase H-Reverse Transcriptase. The levels of specific cDNAs were quantified by PCR-based fluorogenic Taqman assays [34], using FastStart TaqMan ProbeMaster-Rox master mix, primer pairs and the appropriate fluorescent UPL probes as shown in Table 1, in a Mx3005P thermocycler (Stratagene, LaJolla, Calif.). The probes were designed using an online algorithm at the Roche Universal Probe Library Assay Design Center.

TABLE 1

| | | | Amplicon | |
|---|---|---|---|---|
| | Exon Number: Primer Sequence | | Size | Probe |
| Gene | Forward | Reverse | (bp) | # |
| Ldlr | Exon 3: gtcagccgatgcattcct | Exon 4: tcctgggagcacgtcttg | 101 | 80 |
| Pcsk9 | Exon 10: tgcagcatccacaacacc | Exon 11: aaggtcttccacttcccaatg | 114 | 80 |
| Srebp2 | Exon 17: ctacggtgcagagttgct | Exon 18: tcttgatgatctgaggctgga | 72 | 63 |
| Tbp | Exon 1: cggtcgcgtcattttctc | Exon 2: gggttatcttcacacaccatga | 63 | 107 |

Standard curves were established using varying amounts of purified and quantified cDNA amplicons of each mRNA. The level of mRNA for the TATA-binding protein (TBP) was used for normalization.

qRT-PCR

For the mouse studies, the levels of specific mRNAs were quantified in a PCR-based fluorogenic assay using the Taqman technology (Holland et al., 1991). Briefly, total RNA was extracted using the RNeasy extraction kit and reverse-transcribed into cDNA using random hexameric primers and the Superscript II RNase H-Reverse Transcriptase. The cDNA was used as a template to produce PCR amplicons using FastStart TaqMan ProbeMaster-Rox master mix, primer pairs and the appropriate fluorescent probes) in the Stratagene Mx3005P thermocycler. Standard curves were established using varying amounts of pre-quantified amplicons of each transcript. The level of mRNA for the TATA-box binding protein (TBP) was used for normalization.

ELISA

The assays for PCSK9 and insulin were conducted as prescribed by kit manufacturers, using a Thermo Scientific plate reader. For example, PCSK9 levels in conditioned media were measured using the human PCSK9 ELISA kit from Circulex, as specified the manufacturer. The assay was a sandwich immunoassay using two antibodies (A and B) recognizing different PCSK9 epitopes. Briefly, aliquot of diluted media were overlaid on wells coated with anti-PCSK9 antibody A. After 1-h incubation, the wells were washed, overlaid with a solution of HRP-conjugated anti-PCSK9 antibody B, and incubated for 1 h. They were washed again, and overlaid with a solution of tetra-methylbenzidine as a chromogenic substrate for HRP. After 15 min, the reaction was stopped with ammonium sulfate and the absorbance of the reaction mixtures measured by spectrophotometry at 450 nm. All the steps were performed at room temperature. Standards consisted of recombinant human PCSK9.

Immunoblotting

Cell lysates were fractionated by SDS-PAGE and electrophoretically transferred onto a polyvinylidene fluoride membrane. The membrane was incubated with a goat antihuman LDLR, rabbit anti-PCSK9, or rabbit anti-SREBP-2 polyclonal antibody at 1:1000, 1:1500, and 1:200 dilutions, respectively, and then with a HRP-conjugated heterospecific secondary antibody against the primary Igs at a 1:2000 dilution. It was probed for HRP reaction using the Western Lightning Chemiluminescence Reagent Plus a chemiluminescence-based revelation kit. The signal was captured on X-ray film and immunoreactive bands analyzed by densitometry on a Syngene's ChemiGenius²XE Bio Imaging System (Cambridge, Mass.) within the dynamic range of the instrument. The membrane was stripped and reprobed with the anti-β-actin monoclonal primary antibody at 1:20,000 dilution and HRP-conjugated rabbit anti-mouse IgG secondary antibody at a 1:5000 dilution. The densitometric values of β-actin bands were using for normalization of experimental samples.

Immunoprecipitation

Radioactive conditioned media or cell lysates (0.1 ml) were supplemented with 2 l of normal rabbit serum and 15 μl of a 50% (w/v) suspension of Protein A-agarose. After a 1-h incubation at 4° C. with rotational mixing, the samples were centrifuged at 3,000 g for 5 min at 4° C. Supernatants were supplemented 2 μl of rabbit anti-PCSK9 [35], and incubated as above. The resin with bound immune complexes was then sedimented by centrifugation as above, rinsed three times with RIPA buffer, twice with a buffer containing 1 M NaCl, 10 mM Tris-HCl and 1 mM EDTA, pH 8, and twice with PBS containing 1 mM EDTA. Pellets were suspended in 25 μl of 1× Laemmli buffer each, boiled for 5 min, and sedimented as above. Supernatant was subjected to electrophoresis through polyacrylamide gels (8 or 12%). Gels were fixed for 30 min in a 50% methanol-10% acetic acid solution, treated for 30 min with Amplify fluor solution, dried under vacuum and exposed to phosphorimaging screen overnight. Specific radioactive protein bands were visualized and quantified on a Typhoon Phosphorimager (Molecular Dynamics).

GSIS Assay

Cells were seeded and grown to 80% confluence. Prior to GSIS assay, fresh medium containing 3 mM Glucose and 10% FBS medium (low-glucose medium or LGM) without or with Q3G was substituted and incubation resumed for 6 h to adapt the cells to low glucose. Fresh LGM without or with Q3G was substituted and supplemented or not with glucose to the final concentration of 18 mM. After 30 min of incubation, media were collected as above for insulin-specific ELISA.

### Example 1

### Q3G Increases LDLR Expression, While Reducing PCSK9 Secretion

Huh7 cells, hepatocyte derived cellular carcinoma cells, were incubated for 24 h in medium containing 10% lipoprotein-depleted serum (LPDS) and 0 to 10 μM Q3G. The level of LDLR mRNA was measured by quantitative real-time RT-PCR; that of the LDLR protein by semi-quantitative immunoblotting. Exposure to Q3G increased the intracellular content of LDLR mRNA in a concentration-dependent manner; the increase reached a 2× maximum at 2 μM (P<0.01, relative to no Q3G) (FIG. 3A). The content of the corresponding protein followed a similar pattern, but reached a 4× maximum at 4 μM (P<0.005) (FIG. 3B).

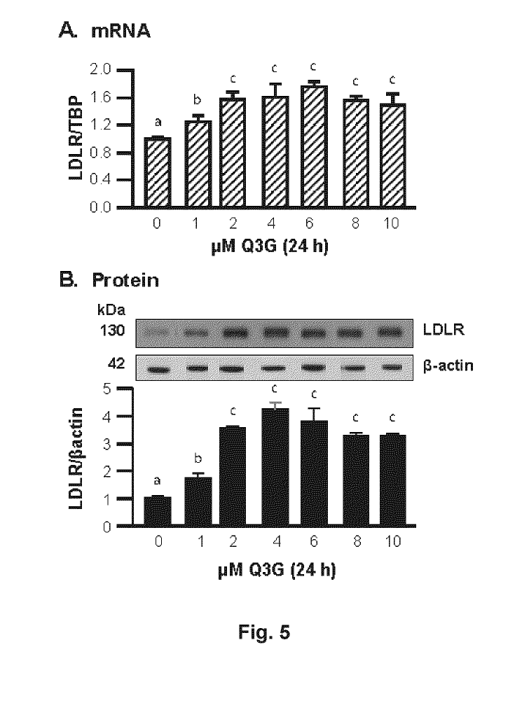

In contrast, at the highest Q3G concentration tested, PCSK9 mRNA levels decreased by one-third (P<0.05) (FIG. 4A), while the levels of the cognate protein increased 1.9× in the cells (P<0.05) (FIG. 4B(a)), and decreased by 35% in conditioned media (P<0.0001) (FIG. 4B(b)), suggesting intracellular retention. At the concentrations used above, the aglycone form of quercetin failed to affect PCSK9 secretion. Furthermore, high Q3G concentrations (>20 NM) attenuated the stimulation of LDLR expression in a concentration-dependent manner (FIG. 5).

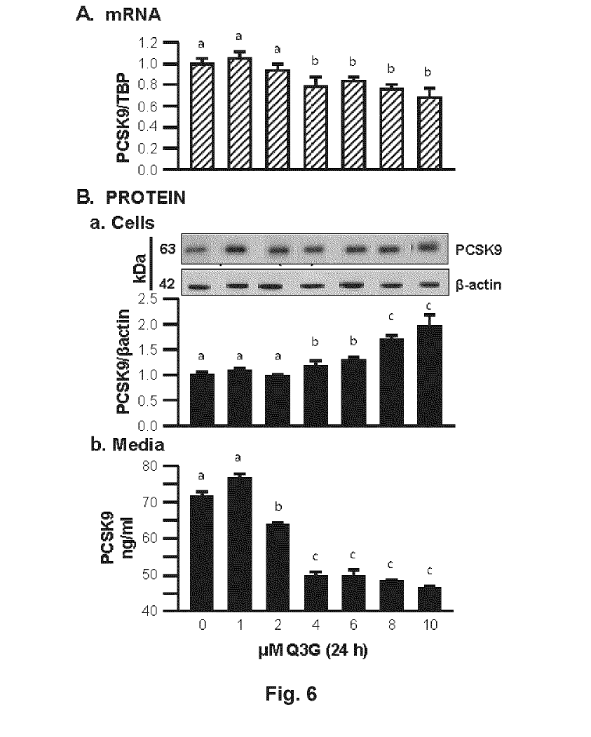

The kinetics of cellular accumulation of LDLR and PCSK9 at 2 μM Q3G was also examined: PCSK9 accumulation in the cells began after a 3-h lag; that of LDLR after 6-h lag (FIG. 6). The longer lag for the receptor suggested that its accumulation might have resulted in part from intracellular retention of the convertase, i.e. of its reduced secretion.

### Example 2

### Q3G Increases ProSREBP-2 Proteolytic Activation

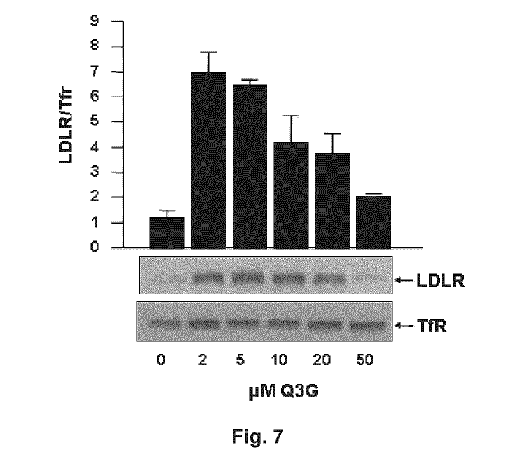

The increase in LDLR mRNA content could be attributed to increased transcription of its gene. This transcription is known to be up regulated by SREBP-2 [36], a nuclear transcriptional factor generated through two successive cleavages of its ER membrane bound precursor, proSREBP-2, by the Golgi proteases PCSK8/S1 P and S2P [37]. We therefore examined the effect of Q3G on SREBP-2 expression. The results are shown in FIG. 7. The flavonoid had no effect on the level of SREBP-2 mRNA (FIG. 7A), but it increased up to 4-fold the ratio of the 65-kDa nuclear form over its 148-kDa ER precursor, indicating stimulated processing of the latter (FIG. 7B). More nuclear SREBP-2 would induce more transcription of the LDLR gene, and account for the increase the intracellular level of its mRNA. The PCSK9 gene promoter can also be activated by SREBP-2 [14,38]. This appeared not be the case in the presence of Q3G, since a decrease in the steady-state level of its mRNA was observed (see FIG. 4A).

### Example 3

### Q3G Delays PCSK9 Secretion

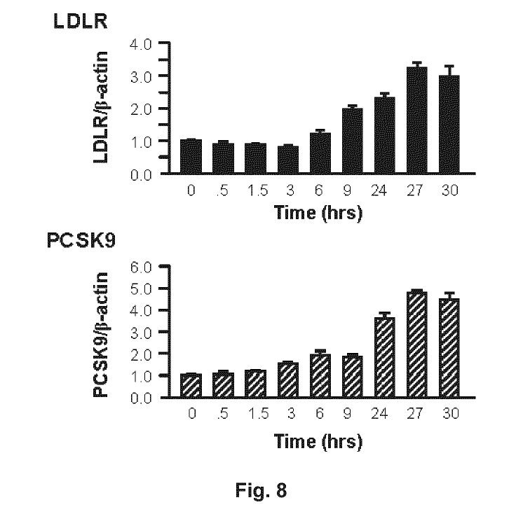

Since PCSK9 can be secreted only after endoproteolytic cleavage of its precursor at the carboxyl end of the prodomain, and the formation of a PCSK9/prosegment complex, it was possible that the reduced secretion of PCSK9 by Q3G-treated Huh7 cells resulted from impaired processing of its precursor. We verified this possibility by pulse-chase analysis. Cells were incubated for 24 h in the absence, or in the presence 5 μM Q3G; they were then metabolically pulse-labeled using radioactive amino acids; the newly biosynthesized radioactive proteins were chased for varying periods of time; PCSK9-related proteins in cell lysates and media were analyzed by immunoprecipitation, SDS/PAGE, and semi-quantitative phosphorimaging. The results are shown in FIG. 8. Chase of untreated and treated cells revealed a gradual intracellular conversion of proPCSK9 to PCSK9 and prosegment, as well as ΔNT-PCSK9 (FIG. 8A), associated with a gradual appearance of the processing products in the culture media (FIG. 8B). There was no obvious difference in the rate of intracellular precursor processing. However, when PCSK9 accumulation in culture media was expressed as a percent of total PCSK9 proteins (proPCSK9, PCSK9, ΔNT-PCSK9 and prosegment), half-maximum accumulation was reached after 60 min in control cells and after 90 min in Q3G-treated cells (FIG. 8C), indicating that pretreatment with Q3G delays PCSK9 secretion.

### Example 4

### Q3G Reduces Simvastatin-Induced PCSK9 Secretion

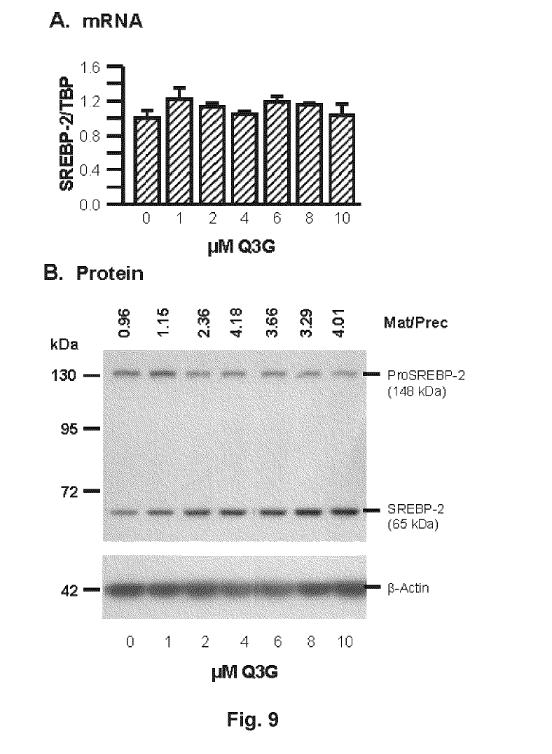

Statins induce expression of LDLR and PCSK9. However, unlike Q3G, they do not reduce PCSK9 secretion. We examined whether, at a 5 μM concentration of Q3G, simvastatin at 0.2 and 1 μM could further up regulate LDLR expression in Huh7 cells; and, inversely, whether the flavonol can reduce statin-stimulated PCSK9 secretion secreted by Huh7 cells. The results are shown in FIG. 9. In the absence of Q3G (open bars), Simvastatin treatment increased, in a concentration-dependent manner, the levels of cellular LDLR (FIG. 9A), cellular PCSK9 (FIG. 9B), and secreted PCSK9 (FIG. 9C). Co-treatment with 5 μM Q3G (black bars), increased cellular LDLR to the level induced by the flavonol alone (FIG. 9A); it further increased the amount of cellular PCSK9 (FIG. 9B), while reducing its level in spent media (FIG. 9C). These results suggested that simvastatin and Q3G stimulated LDLR expression through similar mechanism; but Q3G possessed, in addition, distinct anti-PCSK9 production/secretion properties.

### Example 5

### Q3G Increases Cell Surface Expression of LDLR

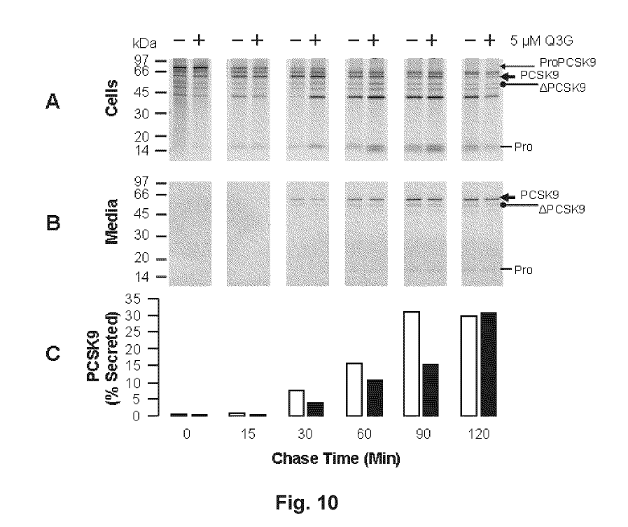

To be functionally relevant, Q3G-induced LDLR should accumulate at the cell surface of the hepatocyte cells where it could mediate LDL uptake. To verify the surface localization of the receptor, untreated and pretreated intact Huh7 cells were stained at 4° C. for LDLR by indirect immunofluorescence, and analyzed by fluorescence flow cytometry. The results are shown in FIG. 10. Pretreatment with Q3G significantly increased (1.7-fold, P<0.001, see histogram) LDLR cell surface density, suggesting that it rendered the hepatocyte cells more capable of taking up more exogenous LDL.

### Example 6

### Q3G Accelerates LDL Uptake

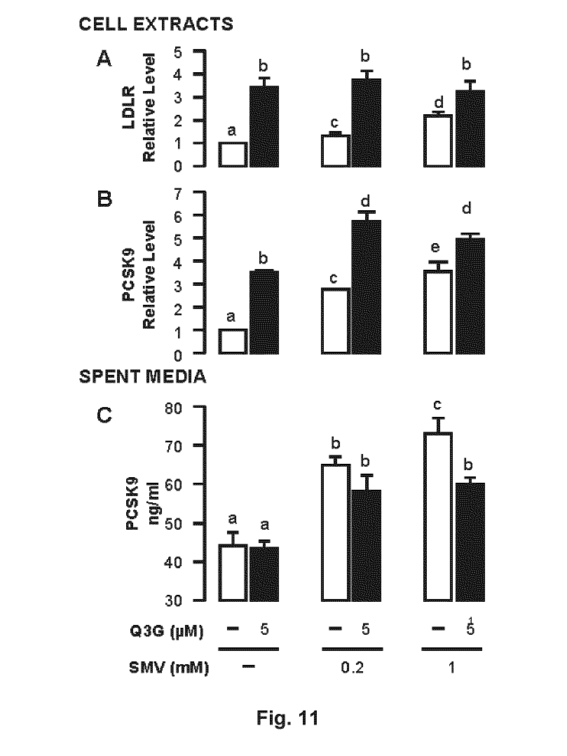

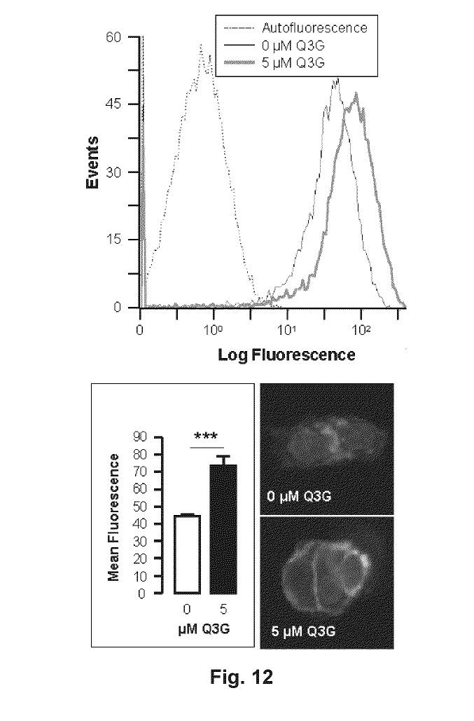

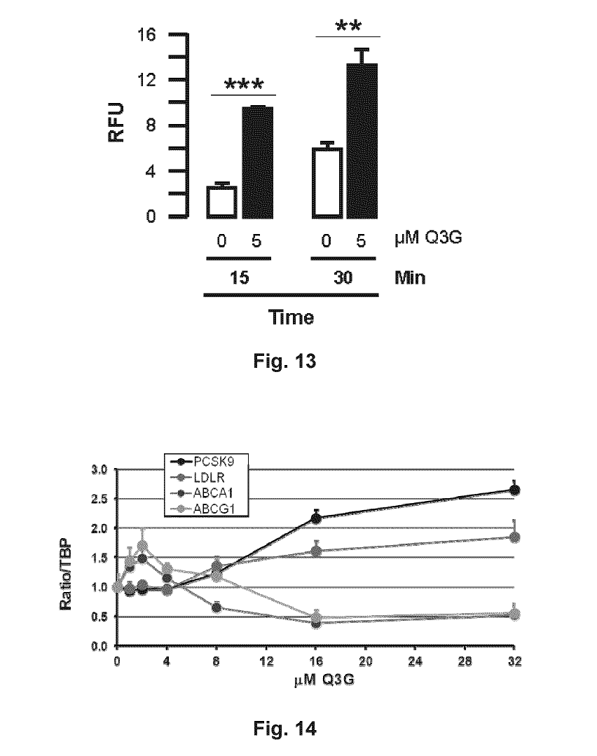

An increase of LDLR expression, combined with a reduction of PCSK9 secretion, should significantly improve the ability of Huh7 cells to take up exogenous LDL. To verify this prediction, cells were incubated overnight in medium supplemented with LPDS to promote expression of the LDLR; they were then treated with 5 μM Q3G for 24 h and exposed to fluorescent bodipy-LDL for 15 or 30 min; after washing, accumulated intracellular LDL was measured by fluorescence spectrometry. As shown in FIG. 11, compared to untreated cells, Q3G-treated cells accumulated 4-fold and 2.5-fold more LDL after 15 min and 30 min, respectively (P<0.005).

### Example 7

### Q3G Increases PCSK9 Expression and Secretion in MIN6 β-Cells

MIN6 β-cells were incubated for 24 h in the presence of different concentrations of Q3G. Total RNA was extracted and analyzed by qRT-PCR for the levels of mRNA for PCSK9, LDLR, ABCA1 and ABCG1. FIG. 14 shows the results, expressed as levels relative to untreated cells.

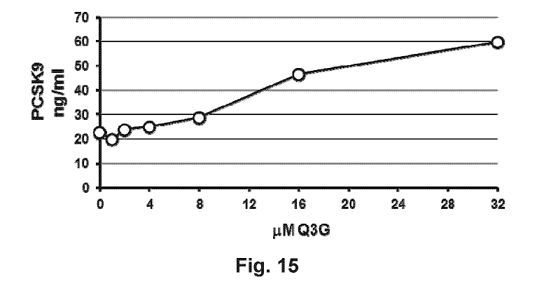

The results indicated that, for pancreatic beta cells, at concentrations of up to 4 mM, Q3G does not affect PCSK9 and LDLR mRNA levels, but increases by about 50% the levels of ABCA1 and ABCG1 mRNA. At 8 mM and above, it increased the mRNA levels of the PCSK9 and LDL-receptor while reducing those of ABCA1 and ABCG1. At the maximum Q3G concentration used (32 mM), the increase in PCSK9 mRNA was greater (2.5-fold than that of LDLR mRNA (1.8-fold). These results suggest that Q3G at low micromolar could promote cholesterol efflux using pancreatic beta cells by increasing the levels of ABCA1 and ABCG1, but at two-digit concentrations, would oppose cholesterol influx into the pancreatic beta cells by increasing more PCSK9 expression and effectively opposing LDLR-mediated uptake of cholesterol. The increase of PCSK9 in the medium paralleled that of the transcript (FIG. 15), indicating a linear correlation between the mRNA translation, protein transport and secretion, i.e. the absence of translation or secretion regulation.

This increased translation is reflected by the higher intracellular content of proPCSK9, presumably located in the endoplasmic reticulum (FIG. 16).

At the protein level, the relative amounts of the LDLR, ABCA1 and ABCG1 in Q3G-treated pancreatic beta cells were in concordance with the relative amounts of mRNA, suggesting that the observed regulation of these cholesterol homeostatic proteins is primarily transcriptional.

### Example 8

### Q3G Does Not Alter GSIS in MIN6 β-Cells

Since exogenous and endogenous cholesterol levels can affect the responsiveness of β-cells secretory granules to exocytosis (Hao et al., 2007; Tsuchiya et al., 2010), the authors of the present disclosure examined whether Q3G regulation of cholesterol homeostatic proteins affected insulin secretion by MIN6 β-cells upon stimulation with 18 mM glucose.

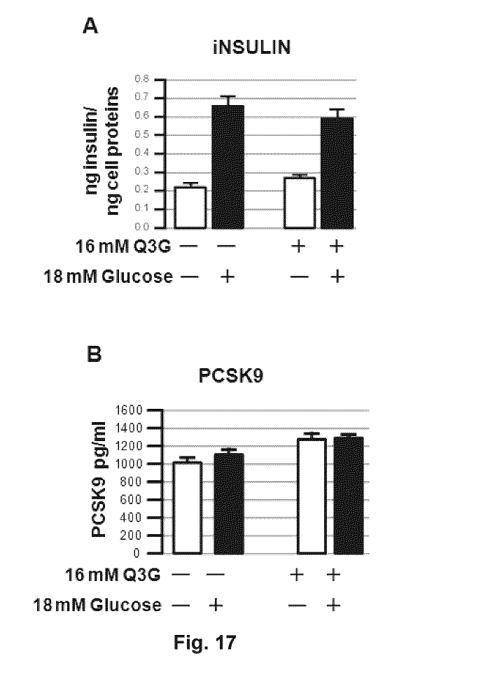

As shown in FIG. 17, stimulated insulin secretion was comparable between untreated and treated cells. Furthermore the level of secreted PCSK9 was unchanged by the stimulation, consistent the intracellular navigation of this protein through the constitutive pathway.

In the preceding description, for purposes of explanation, numerous details are set forth in order to provide a thorough understanding of the examples. However, it will be apparent to one skilled in the art that these specific details are not required.

The above-described examples are intended to be exemplary only. Alterations, modifications and variations can be effected to the particular examples by those of skill in the art without departing from the scope, which is defined solely by the claims appended hereto.

### REFERENCES

1. Havel R J, Kane J P (2001) Structure and metabolism of plasma lipoproteins. In: Scriver C R, Beaudet A L, Sly W S, Valle M D, Childs B et al., editors. The Metabolic & Molecular Basis of Inherited Disease. 8th ed. New York: McGraw-Hill. pp. 2705-2716.
2. Seidah N G, Benjannet S, Wickham L, Marcinkiewicz J, Jasmin S B, et al. (2003) The secretory proprotein convertase neural apoptosis-regulated convertase 1 (NARC-1): Liver regeneration and neuronal differentiation. Proc Natl Acad Sci USA 100: 928-933.
3. Mayne J, Dewpura T, Raymond A, Bernier L, Cousins M, et al. (2011) Novel loss-of function PCSK9 variant is associated with low plasma LDL cholesterol in a French-Canadian family and with impaired processing and secretion in cell culture. Clin Chem 57: 1415-1423.
4. Benjannet S, Rhainds D, Hamelin J, Nassoury N, Seidah N G (2006) The proprotein convertase PCSK9 is inactivated by furin and/or PC5/6A: Functional consequences of natural mutations and post-translational modifications. J Biol Chem 281: 30561-30572.
5. Dewpura T, Raymond A, Hamelin J, Seidah N G, Mbikay M, et al. (2008) PCSK9 is phosphorylated by a Golgi casein kinase-like kinase ex vivo and circulates as a phosphoprotein in humans. FEBS J 275: 3480-3493.
6. Horton J D, Cohen J C, Hobbs H H (2009) PCSK9: a convertase that coordinates LDL catabolism. J Lipid Res 50 Suppl: S172-177.
7. Essalmani R, Susan-Resiga D, Chamberland A, Abifadel M, Creemers J W, et al. (2011) In vivo evidence that furin from hepatocytes inactivates PCSK9. J Biol Chem 286: 4257-4263.
8. Varret M, Abifadel M, Rabes J P, Boileau C (2008) Genetic heterogeneity of autosomal dominant hypercholesterolemia. Clin Genet 73: 1-13.
9. Chretien M, Seidah N G, Basak A, Mbikay M (2008) Proprotein convertases as therapeutic targets. Exp Op Ther Targets 12: 1289-1300.
10. Wild S, Roglic G, Green A, Sicree R, King H (2004) Global prevalence of diabetes: estimates for the year 2000 and projections for 2030. Diabetes Care 27: 1047-1053.
11. Yusuf S, Reddy S, Ounpuu S, Anand S (2001) Global burden of cardiovascular diseases: Part II: variations in cardiovascular disease by specific ethnic groups and geographic regions and prevention strategies. Circulation 104: 2855-2864.
12. Delahoy P J, Magliano D J, Webb K, Grobler M, Liew D (2009) The relationship between reduction in low-density lipoprotein cholesterol by statins and reduction in risk of cardiovascular outcomes: an updated meta-analysis. Clin Ther 31: 236-244.
13. Beltowski J, Wojcicka G, Jamroz-Wisniewska A (2009) Adverse effects of statins—mechanisms and consequences. Curr Drug Saf 4: 209-228.
14. Dubuc G, Chamberland A, Wassef H, Davignon J, Seidah N G, et al. (2004) Statins upregulate PCSK9, the gene encoding the proprotein convertase neural apoptosis-regulated convertase-1 implicated in familial hypercholesterolemia. Arterioscl, Thromb, Vasc Biol 24: 1454-1459.
15. Mayne J, Dewpura T, Raymond A, Cousins M, Chaplin A, et al. (2008) Plasma PCSK9 levels are significantly modified by statins and fibrates in humans. Lipids Health Dis 7: 22.
16. Careskey H E, Davis R A, Alborn W E, Troutt J S, Cao G, et al. (2007) Atorvastatin increases human serum levels of proprotein convertase subtilisin kexin type 9 (PCSK9). J Lipid Res.
17. Welder G, Zineh I, Pacanowski M A, Troutt J S, Cao G, et al. (2010) High-dose atorvastatin causes a rapid sustained increase in human serum PCSK9 and disrupts its correlation with LDL cholesterol. J Lipid Res 51: 2714-2721.
18. Rashid S, Curtis D E, Garuti R, Anderson N N, Bashmakov Y, et al. (2005) Decreased plasma cholesterol and hypersensitivity to statins in mice lacking Pcsk9. Proceedings of the National Academy of Sciences of the United States of America 102: 5374-5379.
19. Berge K E, Ose L, Leren T P (2006) Missense mutations in the PCSK9 Gene are associated with hypocholesterolemia and possibly increased response to statin therapy. Arterioscl Thromb Vasc Biol 26: 1094-1100.
20. Pisciotta L, Sallo R, Rabacchi C, Wunsch A, Calandra S, et al. (2011) Leucine 10 allelic variant in signal peptide of PCSK9 increases the LDL cholesterol-lowering effect of statins in patients with familial hypercholesterolaemia. Nutr Metab Cardiovasc Dis.
21. Seidah N G (2009) PCSK9 as a therapeutic target of dyslipidemia. Expert Opin Ther Targets 13: 19-28.
22. Mukherjee P K (2003) Plant products with hypocholesterolemic potentials. Adv Food Nutr Res 47: 277-338.
23. Chumark P, Khunawat P, Sanvarinda Y, Phornchirasilp S, Morales N P, et al. (2008) The in vitro and ex vivo antioxidant properties, hypolipidaemic and antiatherosclerotic activities of water extract of Moringa oleifera Lam. leaves. J Ethnopharmacol 116: 439-446.
24. Ghasi S, Nwobodo E, Ofili J O (2000) Hypocholesterolemic effects of crude extract of leaf of Moringa oleifera Lam in high-fat diet fed wistar rats. J Ethnopharmacol 69: 21-25.
25. Nambiar V S, Guin P, Parnami S, Daniel M (2010) Impact of antioxidants from drumstick leaves on the lipid profile of hyperlipidemics. J Herbal Med Toxicol 4: 165-172.
26. Kumari D J (2010) Hypglycemic effect of Moringa oleifera and Azadichta indica in type-2 diabetes. The Bioscan 5: 211-214.
27. Mbikay M (2012) Therapeutic potential of Moringa oleifera leaves in chronic hyperglycemia and dyslipidemia: a review. Front Pharmacol 3: 24.
28. Lako J, Trenerry V C, Wahlqvist M, Wattanapenpaiboon N, Sotheeswaran S, et al. (2007) Phytochemical flavonols, carotenoids and the antioxidant properties of a wide selection of Fijian fruit, vegetables and other readily available foods. Food Chem 101: 1727-1741.
29. Bennett R N, Mellon F A, Foidl N, Pratt J H, Dupont M S, et al. (2003) Profiling glucosinolates and phenolics in vegetative and reproductive tissues of the multipurpose trees Moringa oleifera L. (horseradish tree) and Moringa stenopetala L. J Agric Food Chem 51: 3546-3553.
30. Atawodi S E, Atawodi J C, Idakwo G A, Pfundstein B, Haubner R, et al. (2010) Evaluation of the polyphenol content and antioxidant properties of methanol extracts of the leaves, stem, and root barks of Moringa oleifera Lam. J Med Food 13: 710-716.
31. Juzwiak S, Wojcicki J, Mokrzycki K, Marchlewicz M, Bialecka M, et al. (2005) Effect of quercetin on experimental hyperlipidemia and atherosclerosis in rabbits. Pharmacol Rep 57: 604-609.
32. Kamada C, da Silva E L, Ohnishi-Kameyama M, Moon J H, Terao J (2005) Attenuation of lipid peroxidation and hyperlipidemia by quercetin glucoside in the aorta of high cholesterol-fed rabbit. Free Radic Res 39: 185-194.
33. Rivera L, Moron R, Sanchez M, Zarzuelo A, Galisteo M (2008) Quercetin ameliorates metabolic syndrome and improves the inflammatory status in obese Zucker rats. Obesity 16: 2081-2087.
34. Holland P M, Abramson R D, Watson R, Gelfand D H (1991) Detection of specific polymerase chain reaction product by utilizing the 5′ - - - 3′ exonuclease activity of Thermus aquaticus DNA polymerase. Proc Natl Acad Sci USA 88: 7276-7280.
35. Mayne J, Raymond A, Chaplin A, Cousins M, Kaefer N, et al. (2007) Plasma PCSK9 levels correlate with cholesterol in men but not in women. Biochem Biophys Res Comm 361: 451-456.
36. Hua X, Yokoyama C, Wu J, Briggs M R, Brown M S, et al. (1993) SREBP-2, a second basic-helix-loop-helix-leucine zipper protein that stimulates transcription by binding to a sterol regulatory element. Proc Natl Acad Sci USA 90: 11603-11607.
37. Brown M S, Goldstein J L (1999) A proteolytic pathway that controls the cholesterol content of membranes, cells, and blood. Proc Natl Acad Sci USA 96: 11041-11048.
38. Jeong H J, Lee H S, Kim K S, Kim Y K, Yoon D, et al. (2008) Sterol-dependent regulation of proprotein convertase subtilisin/kexin type 9 expression by sterolregulatory element binding protein-2. J Lipid Res 49: 399-409.
39. Poirier S, Mayer G, Poupon V, McPherson P S, Desjardins R, et al. (2009) Dissection of the endogenous cellular pathways of PCSK9-induced low density lipoprotein receptor degradation: evidence for an intracellular route. J Biol Chem 284: 28856-28864.
40. Chan J C, Piper D E, Cao Q, Liu D, King C, et al. (2009) A proprotein convertase subtilisin/kexin type 9 neutralizing antibody reduces serum cholesterol in mice and nonhuman primates. Proc Natl Acad Sci USA 106: 9820-9825.
41. Ni Y G, Di Marco S, Condra J H, Peterson L B, Wang W, et al. (2011) A PCSK9-binding antibody that structurally mimics the EGF(A) domain of LDL-receptor reduces LDL cholesterol in vivo. J Lipid Res 52: 78-86.
42. Li H, Dong B, Park S W, Lee H S, Chen W, et al. (2009) Hepatocyte nuclear factor 1alpha plays a critical role in PCSK9 gene transcription and regulation by the natural hypocholesterolemic compound berberine. J Biol Chem 284: 28885-28895.
43. Boulton D W, Walle U K, Walle T (1998) Extensive binding of the bioflavonoid quercetin to human plasma proteins. J Pharm Pharmacol 50: 243-249.
44. Walle T, Vincent T S, Walle U K (2003) Evidence of covalent binding of the dietary flavonoid quercetin to DNA and protein in human intestinal and hepatic cells. Biochem Pharmacol 65: 1603-1610.
45. Kaldas M I, Walle U K, van der Woude H, McMillan J M, Walle T (2005) Covalent binding of the flavonoid quercetin to human serum albumin. J Agric Food Chem 53: 4194-4197.
46. Sun H, Samarghandi A, Zhang N, Yao Z, Xiong M, et al. (2012) Proprotein convertase subtilisin/kexin type 9 interacts with apolipoprotein B and prevents its intracellular degradation, irrespective of the low-density lipoprotein receptor. Arterioscler Thromb Vasc Biol.
47. Casaschi A, Wang Q, Dang K, Richards A, Theriault A (2002) Intestinal apolipoprotein B secretion is inhibited by the flavonoid quercetin: potential role of microsomal triglyceride transfer protein and diacylglycerol acyltransferase. Lipids 37: 647-652.
48. Moon J, Lee S M, Do H J, Cho Y, Chung J H, et al. (2012) Quercetin up-regulates LDL receptor expression in HepG2 Cells. Phytother Res 26: 1688-1694.
49. Cermak R, Landgraf S, Wolffram S (2003) The bioavailability of quercetin in pigs depends on the glycoside moiety and on dietary factors. J Nutr 133: 2802-2807.
50. Reinboth M, Wolffram S, Abraham G, Ungemach F R, Cermak R (2010) Oral bioavailability of quercetin from different quercetin glycosides in dogs. Br J Nutr 104: 198-203.
51. Cermak R, Landgraf S, Wolffram S (2004) Quercetin glucosides inhibit glucose uptake into brush-border-membrane vesicles of porcine jejunum. Br J Nutr 91: 849-855.
52. Bischoff S C (2008) Quercetin: potentials in the prevention and therapy of disease. Curr Opin Clin Nutr Metab Care 11: 733-740.
53. Notas G, Nifli A P, Kampa M, Pelekanou V, Alexaki V I, et al. (2011) Quercetin accumulates in nuclear structures and triggers specific gene expression in epithelial cells. J Nutr Biochem 23: 656-666

What is claimed is:

1. Use of quercetin-3-O-β-D-glucoside (Q3G) for increasing the amount of cell surface low-density lipoprotein receptor (LDLR) on a hepatocyte cell and reducing the amount of functional proprotein convertase subtilisin/kexin type 9 (PCSK9) secreted by the hepatocyte cell,
wherein the Q3G is formulated for administration to the hepatocyte cell, and
wherein the increase in cell surface LDLR and the decrease in secretion of functional PCSK9 is in comparison to the hepatocyte cell not exposed to Q3G.

2. The use according to claim 1, wherein the Q3G is formulated for administration to provide a concentration of Q3G at the hepatocyte cell, in the extracellular medium, between about 0.1 μM and about 100 NM.

3. The use according to claim 1 or 2, wherein the Q3G is formulated for administration to a patient having dyslipidemia and the increased amount of cell surface LDLR on the hepatocyte cell and the reduced amount of functional PCSK9 secreted by the hepatocyte cell is for treating metabolic syndrome, or a hypercholesterolemia related-disease or disorder.

4. The use according to claim 3, wherein the hypercholesterolemia related-disease or disorder is an obesity-related disease, atherosclerosis, coronary artery disease, stroke, or type 2 diabetes.

5. The use according to claim 3 or 4 wherein the Q3G is formulated for oral administration.

6. Use of quercetin-3-O-β-D-glucoside (Q3G) for reducing the amount of cell surface low-density lipoprotein receptor (LDLR) on a pancreatic beta cell and increasing the amount of functional proprotein convertase subtilisin/kexin type 9 (PCSK9) secreted by the pancreatic beta cell,
wherein the Q3G is formulated for administration to the pancreatic beta cell, and
wherein the decrease in cell surface LDLR and the increase in secretion of functional PCSK9 is in comparison to the pancreatic beta cell not exposed to Q3G.

7. The use according to claim 6, wherein the Q3G is formulated for administration to provide a concentration of Q3G at the pancreatic beta cell, in the extracellular medium, between about 4 μM and about 100 NM.

8. The use according to claim 6 or 7, wherein the Q3G is formulated for administration to a patient having dyslipidemia and the decreased amount of cell surface LDLR on the pancreatic beta cell and the increased amount of functional PCSK9 secreted by the pancreatic beta cell is for reducing cytotoxic effects associated with cholesterol uptake by the pancreatic beta cell.

9. The use according to claim 8, wherein the hypercholesterolemia related-disease or disorder is an obesity-related disease, atherosclerosis, coronary artery disease, stroke, or type 2 diabetes.

10. The use according to claim 8 or 9 wherein the Q3G is formulated for oral administration.

11. Use of quercetin-3-O-β-D-glucoside (Q3G) in combination with a statin for increasing the amount of cell surface low-density lipoprotein receptor (LDLR) on a hepatocyte cell and reducing the amount of functional proprotein convertase subtilisin/kexin type 9 (PCSK9) secreted by the hepatocyte cell,
wherein the Q3G and the statin are formulated for administration to the hepatocyte cell,
wherein the increase in cell surface LDLR is in comparison to the hepatocyte cell not exposed to either the Q3G or the statin, and
wherein the decrease in secretion of functional PCSK9 is in comparison to the hepatocyte cell exposed to the statin but not exposed to Q3G.

12. The use according to claim 11, wherein the Q3G is formulated for administration to provide a concentration of Q3G at the hepatocyte cell, in the extracellular medium, between about 0.1 μM and about 100 NM.

13. The use according to claim 11 or 12, wherein the statin is simvastatin.

14. The use according to any one of claims 11 to 13, wherein the Q3G and the statin are formulated for administration to a patient having dyslipidemia and the increased amount of cell surface LDLR on the hepatocyte cell and the reduced amount of functional PCSK9 secreted by the hepatocyte cell is for treating metabolic syndrome, or a hypercholesterolemia related-disease or disorder.

15. The use according to claim 14, wherein the hypercholesterolemia related-disease or disorder is an obesity-related disease, atherosclerosis, coronary artery disease, stroke, or type 2 diabetes.

16. The use according to claim 14 or 15, wherein the Q3G is formulated for oral administration.

17. A composition comprising quercetin-3-O-β-D-glucoside (Q3G) and a statin, the composition for increasing the amount of cell surface low-density lipoprotein receptor (LDLR) on a hepatocyte cell and reducing the amount of functional proprotein convertase subtilisin/kexin type 9 (PCSK9) secreted by the hepatocyte cell,
wherein the increase in cell surface LDLR is in comparison to the hepatocyte cell not exposed to either the Q3G or the statin, and
wherein the decrease in secretion of functional PCSK9 is in comparison to the hepatocyte cell exposed to the statin but not exposed to Q3G.

18. The composition according to claim 17 wherein the statin is simvastatin.

19. A composition comprising quercetin-3-O-β-D-glucoside (Q3G) and a statin, the composition for:
increasing the amount of cell surface low-density lipoprotein receptor (LDLR) on a hepatocyte cell and reducing the amount of functional proprotein convertase subtilisin/kexin type 9 (PCSK9) secreted by the hepatocyte cell, wherein the increase in cell surface LDLR is in comparison to the hepatocyte cell not exposed to either the Q3G or the statin, and wherein the decrease in secretion of functional PCSK9 is in comparison to the hepatocyte cell exposed to the statin but not exposed to Q3G; and
reducing the amount of cell surface low-density lipoprotein receptor (LDLR) on a pancreatic beta cell and increasing the amount of functional proprotein convertase subtilisin/kexin type 9 (PCSK9) secreted by the pancreatic beta cell, wherein the decrease in cell surface LDLR is in comparison to the pancreatic beta cell not exposed to either the Q3G or the statin, and wherein the increase in secretion of functional PCSK9 is in comparison to the pancreatic cell exposed to the statin but not exposed to Q3G.

20. A method of increasing the amount of cell surface low-density lipoprotein receptor (LDLR) on a hepatocyte cell and reducing the amount of functional proprotein convertase subtilisin/kexin type 9 (PCSK9) secreted by the hepatocyte cell, the method comprising:
treating the hepatocyte cell with an effective concentration of quercetin-3-O-β-D-glucoside (Q3G) the increase in cell surface LDLR and the decrease in secretion of functional PCSK9 being in comparison to the hepatocyte cell prior to treatment with the Q3G.

21. The method according to claim 20 wherein the effective concentration of Q3G at the hepatocyte cell, in the extracellular medium, between about 0.1 μM and about 100 μM.

22. A method of not substantially changing, or of decreasing the amount of cell surface low-density lipoprotein receptor (LDLR) on a pancreatic beta cell, and increasing the amount of functional proprotein convertase subtilisin/kexin type 9 (PCSK9) secreted by the pancreatic beta cell, the method comprising:
treating the pancreatic beta cell with an effective concentration of quercetin-3-O-β-D-glucoside (Q3G) the increase in cell surface LDLR and the decrease in secretion of functional PCSK9 being in comparison to the pancreatic beta cell prior to treatment with the Q3G.

23. The method according to claim 22 wherein the effective concentration of Q3G at the pancreatic beta cell, in the extracellular medium, between about 4 μM and about 100 μM.

24. A method of increasing the amount of cell surface low-density lipoprotein receptor (LDLR) on a hepatocyte cell and reducing the amount of functional proprotein convertase subtilisin/kexin type 9 (PCSK9) secreted by the hepatocyte cell, the method comprising:
treating the hepatocyte cell with an effective amount of quercetin-3-O-β-D-glucoside (Q3G) and a statin,
the increase in cell surface LDLR being in comparison to the hepatocyte cell not exposed to either the Q3G or the statin, and
the decrease in secretion of functional PCSK9 being in comparison to the hepatocyte cell exposed to the statin but not exposed to Q3G.

25. A method of reducing plasma cholesterol levels in a patient in need thereof, the method comprising:
administering to the patient a therapeutically effective amount of quercetin-3-O-β-D-glucoside (Q3G) to increase the amount of cell surface low-density lipoprotein receptor (LDLR) on a hepatocyte cell and to reduce the amount of functional proprotein convertase subtilisin/kexin type 9 (PCSK9) secreted by the hepatocyte cell, thereby increasing rate of cellular uptake of exogenous LDL from the plasma of the patient and reducing the plasma cholesterol levels in the patient, the increase in cell surface LDLR and the decrease in secretion of functional PCSK9 being in comparison to the hepatocyte cell prior to exposure to the Q3G.

26. The method according to claim 25, wherein administration of the Q3G increases the amount of functional PCSK9 secreted by a pancreatic beta cell and decreases or not substantially change the amount of cell surface LDLR on the pancreatic beta cell, the decrease or lack of substantial change in cell surface LDLR and the increase in secretion of functional PCSK9 being in comparison to the pancreatic beta cell prior to exposure to the Q3G.

27. The method according to claim 25 or 26, wherein the reduction of plasma cholesterol results in the treatment or prevention of metabolic syndrome, or a hypercholesterolemia related-disease or disorder.

28. The method according to claim 27, wherein the hypercholesterolemia related-disease or disorder is an obesity-related disease, atherosclerosis, coronary artery disease, stroke, or type 2 diabetes.

29. The method according to any one of claims 25 to 28, wherein administering Q3G to the patient is orally administering Q3G to the patient.

30. A method of reducing plasma cholesterol levels in a patient in need thereof, the method comprising:
administering to the patient a therapeutically effective amount of quercetin-3-O-β-D-glucoside (Q3G) and a therapeutically effective amount of a statin;
wherein treatment of the patient with the Q3G and the statin increases the amount of cell surface low-density lipoprotein receptor (LDLR) on a hepatocyte cell when compared to the hepatocyte cell not exposed to either the Q3G or the statin, and reduces the amount of functional proprotein convertase subtilisin/kexin type 9 (PCSK9) secreted by the hepatocyte cell in comparison to the hepatocyte cell exposed to the statin but not exposed to Q3G,
the increased amount of hepatocyte cell surface LDLR and reduced amount of functional PCSK9 secreted by the hepatocyte cell resulting in an increased rate of cellular uptake of exogenous LDL from the plasma of the patient and a reduced level of plasma cholesterol in the patient.

31. The method according to claim 30, wherein treatment of the patient with the Q3G increases the amount of functional PCSK9 secreted by a pancreatic beta cell and decreases the amount of cell surface LDLR on the pancreatic beta cell, the decrease in cell surface LDLR and the increase in secretion of functional PCSK9 being in comparison to the pancreatic beta cell prior to exposure to the Q3G.

32. The method according to claim 30 or 31, wherein the reduction of plasma cholesterol results in the treatment or prevention of metabolic syndrome, or a hypercholesterolemia related-disease or disorder.

33. The method according to claim 32, wherein the hypercholesterolemia related-disease or disorder is an obesity-related disease, atherosclerosis, coronary artery disease, stroke, or type 2 diabetes.

34. The method according to any one of claims 30 to 33, wherein administering Q3G to the patient is orally administering Q3G to the patient.
